(19) United States
(12) Patent Application Publication
(10) Pub. No.: US 2014/0324009 A1
(43) Pub. Date: Oct. 30, 2014

(54) Aperture-Patterned Fibrous Nonwoven Web

(71) Applicant: KIMBERLY-CLARK WORLDWIDE, INC., NEENAH, WI (US)

(72) Inventors: JuHyung Lee, Gyeonggi-do (KR); HyungByum Kim, Gyeonggi-do (KR); HyungWoo Park, GyeongGi-Do (KR); SeongDae Roh, Gyeonggi-do (KR); SeoYeon Son, Gyeonggi-do (KR); SungSu A. Kim, Gyeonggi-do (KR); Xie Jihua, Xiamen City (CN)

(73) Assignee: KIMBERLY-CLARK WORLDWIDE, INC., NEENAH, WI (US)

(21) Appl. No.: 14/258,680

(22) Filed: Apr. 22, 2014

Related U.S. Application Data
(60) Provisional application No. 61/817,009, filed on Apr. 29, 2013.

(51) Int. Cl. A61F 13/538

(52) U.S. Cl. CPC A61F 13/538
USPC 604/384; 428/131; 428/137

(57) ABSTRACT

An apertured fibrous nonwoven web with an array of apertures is made that allows for increased functionality and improved aesthetic appearance. The material is particularly well-suited for use as a topsheet or bodyside liner material for personal care absorbent articles including, but not limited to feminine hygiene products, diapers, diaper pants, training pants, adult incontinence devices, bandages and the like. The fibrous nonwoven web includes first, second and third arrays of apertures with varying characteristics that provide improved fluid handling properties for various body exudates.

This application claims the benefit of priority from U.S. Provisional Application No. 61/817,009 filed on Apr. 29, 2013.

### FIELD OF THE INVENTION

The present invention relates to apertured and embossed fibrous nonwoven webs which are particularly well adapted for use as intake layers for personal care absorbent articles including, but not limited to, feminine hygiene products. The present invention further relates to equipment and a process for forming such apertured and embossed fibrous nonwoven webs.

### BACKGROUND OF THE INVENTION

Fibrous nonwoven webs are used in a wide variety of applications many of which are single or limited use before disposal takes place. While such nonwoven webs are typically liquid permeable, in certain applications greater permeability is required and so aperturing of the web is also undertaken. This is particularly true with absorbent articles such as personal care absorbent articles including, but not limited to diapers, diaper pants, training pants, adult incontinence products, feminine hygiene products, bandages and the like. In such applications, such fibrous nonwoven webs are often used as bodyside liner materials (also referred to as the topsheet) and/or one or more of the underlying layers.

In many of these applications, feminine hygiene products being a prime example, for such a fibrous nonwoven web to be successful, it should attempt to satisfy a number of criteria. A topsheet in a sanitary napkin is one of the most important materials affecting the consumer's purchasing intention because it is a very critical factor in determining the product appearance and performance. Over the years, consumers' needs and uses for feminine hygiene products have become greater and more complicated. As a result, it has become increasingly more important to differentiate products from one another as to performance, appearance and aesthetic appeal. For this reason, many studies have been conducted relative to the product's overall appearance and performance and specifically the topsheet construction including its fiber composition, degree of perforation, embossing design and post-treatment relative to such attributes as softness, feel, fluid intake, rewet and other performance characteristics. Perforation technology is widely used to provide improved absorbency and absorbent visual cues, and embossing patterns are used to provide improved breathability by lessening the skin contact surface area. However, many of the commercialized embossing patterns are very simple, linear designs, that are not highly differentiated by the consumers and tend to perform the same.

To make a more three-dimensional and visually distinctive pattern, multi-step perforation, embossing and lamination steps are often employed. Practically, in such situations, higher basis weight materials are required to make visually attractive embossing. Further, to make embossing and perforation patterns in the same product, multi-step processes are generally required. For example, often the fibrous nonwoven web is perforated in a first step and then laminated with a non-perforated nonwoven. Next, the combination is typically joined using ultrasonics, adhesives or embossing. Perforation and embossing can be made on the same material in one step without lamination process but it has been found that the embossing pattern can be very easily deformed by the tension and pressure used during the formation process, especially when high basis weight materials are not being used.

There is therefore a need for a fibrous nonwoven web which is pattern embossed and apertured which can be used in a wide variety of applications where such materials are needed for such attributes as fluid intake, improved visual appearance and overall performance. This is particularly the case with fibrous nonwoven webs which are used as topsheets for personal care absorbent articles such as feminine hygiene products including, for example, sanitary napkins. It is an object of the present invention to provide perforated patterns with improved breathability and a more visually distinctive look with no increase in basis weight and process cost, wherein the pattern has a high enough open area to maintain absorbency and breathability and is not easily deformed by the tension and pressure applied during the manufacturing process.

### SUMMARY OF THE INVENTION

Disclosed herein is a fibrous nonwoven web with a top surface and a bottom surface with the web including a first array of first apertures with each of the first apertures residing in a respective first depression in the web. The first depression has a first depression depth, a first depression density, a first depression open area and a first depression shape with the first array of first apertures defining a first aperture density, a first aperture open area and a first aperture shape. The web also includes a second array of second apertures with each of the second apertures residing in a respective second depression in the web. The second depression has a second depression depth, a second depression density, a second depression open area and a second depression shape. The web also includes a second array of second apertures defining a second aperture density, a second aperture open area and a second aperture shape with the second array of second apertures at least partially surrounding the first array. The second aperture depth is different from the first aperture depth, and at least one of the second aperture density and the second aperture open area respectively is different from the first aperture density and the first aperture open area. The web also includes a third array of third apertures, each of the third apertures residing in a respective third depression in the web with the third depression having a third depression depth, a third depression density, a third depression open area and a third depression shape. The third array of third apertures defines a third aperture density, a third aperture open area and a third aperture shape with the second array at least partially separating the first array from the third array.

In another embodiment of the fibrous nonwoven web at least a portion of one of the first, second and third apertures is located in or adjacent the bottom surface and at least another portion of one of the other of the first, second and third apertures is located intermediate the top surface and the bottom surface.

In a further embodiment, the fibrous nonwoven web can have at least one of the first aperture density and the first aperture open area be respectively greater than the second aperture density and the second aperture open area.

In an embodiment, the fibrous nonwoven web can have the first apertures each define a first major axis and the second apertures each define a second major axis with the second major axis being greater than the first major axis.

Also disclosed herein is an absorbent article which can include a topsheet, a backsheet and an absorbent core disposed between the topsheet and the backsheet. The absorbent article can optionally include one or more additional layers disposed between the topsheet and the absorbent core with at least a portion of the absorbent article comprising the aforementioned fibrous nonwoven web.

In another embodiment, an absorbent article is disclosed which includes a topsheet, a backsheet and an absorbent core disposed between the topsheet and the backsheet and optionally one or more additional layers disposed between the topsheet and the absorbent core. In this embodiment at least a portion of the absorbent article can comprise one of the fibrous nonwoven webs described herein with the fibrous nonwoven web including any one or more of the following features: the second aperture depth is different from the first aperture depth; at least one of the second aperture density and the second aperture open area respectively is different from the first aperture density and the first aperture open area; the second array of second apertures at least partially surrounding the first array; the second array at least partially separates the first array from the third array; the second array has a sinusoidal shape; the first array has a noncircular shape; the third array has a sinusoidal shape; the first, second and third arrays form a repeating pattern and the second and third arrays are in phase with one another; the first, second and third arrays form a repeating pattern and the second and third arrays are out-of-phase with one another; at least one of the third aperture density and the third aperture open area is respectively greater than the second aperture density and the second aperture open area; the second array completely surrounds the first array and at least one of the second aperture open area and the second depression open area is respectively less than the first aperture open area and the first depression open area; and the second array completely surrounds the first array and at least one of the second aperture shape and the second depression shape is respectively different from the first aperture shape and the first depression shape.

With any of the absorbent article combinations described herein the article can have a layer subjacent the topsheet which is darker in color than the topsheet to further assist in stain masking of fluids absorbed by the absorbent article.

In any of the fibrous nonwoven webs described herein the second array can have a sinusoidal shape, optionally, the first array can have a noncircular shape and optionally the third array can have a sinusoidal shape. Still further, the noncircular shape can comprise a leaf-like design.

In an embodiment, the fibrous nonwoven can have the first, second and third arrays form a repeating pattern with the second and third arrays being in phase with one another.

In an embodiment, the fibrous nonwoven web can have the first, second and third arrays form a repeating pattern with the second and third arrays being out-of-phase with one another.

In one embodiment the fibrous nonwoven web has at least one of the third aperture density and the third aperture open area designed to be respectively greater than the second aperture density and the second aperture open area.

In another embodiment, the fibrous nonwoven web has the second array completely surrounding the first array and at least one of the second aperture open area and the second depression open area is respectively less than the first aperture open area and the first depression open area.

In still a further embodiment the fibrous nonwoven web is designed such that the second array completely surrounds the first array and at least one of the second aperture shape and the second depression shape is respectively different from the first aperture shape and the first depression shape.

In yet another embodiment, a film layer can be substituted for the fibrous nonwoven webs described herein and the same first, second and third arrays can be utilized with the film.

A further embodiment can include a fibrous nonwoven web with a top surface and a bottom surface with the web including a first array of first apertures with each of the first apertures residing in a respective first depression in the web. The first depression has a first depression depth, a first depression density, a first depression open area and a first depression shape with the first array of first apertures defining a first aperture density, a first aperture open area and a first aperture shape. The web also includes a second array of second apertures with each of the second apertures residing in a respective second depression in the web. The second depression has a second depression depth, a second depression density, a second depression open area and a second depression shape. The web also includes a second array of second apertures defining a second aperture density, a second aperture open area and a second aperture shape with the second array of second apertures at least partially surrounding the first array. The second aperture depth is different from the first aperture depth, and at least one of the second aperture density and the second aperture open area respectively is different from the first aperture density and the first aperture open area. The web also includes a third array of third apertures, each of the third apertures residing in a respective third depression in the web with the third depression having a third depression depth, a third depression density, a third depression open area and a third depression shape. The third array of third apertures defines a third aperture density, a third aperture open area and a third aperture shape with the second array at least partially separating the first array from the third array. Still further, the fibrous nonwoven web can include any one or more of the following optional features: the second aperture depth is different from the first aperture depth; at least one of the second aperture density and the second aperture open area respectively is different from the first aperture density and the first aperture open area; the second array of second apertures at least partially surrounding the first array; the second array at least partially separates the first array from the third array; the second array has a sinusoidal shape; the first array has a noncircular shape; the third array has a sinusoidal shape; the first, second and third arrays form a repeating pattern and the second and third arrays are in phase with one another; the first, second and third arrays form a repeating pattern and the second and third arrays are out-of-phase with one another; at least one of the third aperture density and the third aperture open area is respectively greater than the second aperture density and the second aperture open area; the second array completely surrounds the first array and at least one of the second aperture open area and the second depression open area is respectively less than the first aperture open area and the first depression open area; and the second array completely surrounds the first array and at least one of the second aperture shape and the second depression shape is respectively different from the first aperture shape and the second depression shape.

Also disclosed herein is a fibrous nonwoven laminate comprising a first fibrous nonwoven web and a second fibrous nonwoven web with the second fibrous nonwoven web defining an upper surface and a lower surface. The first fibrous nonwoven web comprises any one of the aforementioned fibrous nonwoven web combinations with the bottom surface of this fibrous nonwoven web positioned adjacent the upper surface of the second fibrous nonwoven web whereby a first air gap defining a first vertical height and a second air gap defining a second vertical height are formed between the first fibrous nonwoven web and the first fibrous nonwoven web.

### DESCRIPTION OF THE DRAWINGS

A full and enabling disclosure of the present invention is set forth more particularly in the remainder of the specification, including reference to the accompanying figures, in which:

FIG. 1 is a top plan view of an absorbent article according to the present invention which utilizes a fibrous nonwoven web according to the present invention.

FIG. 2 is an exploded perspective view of an absorbent article according to the present invention which utilizes a fibrous nonwoven web according to the present invention.

FIG. 3 is a cross-section of an absorbent article according to the present invention which utilizes a fibrous nonwoven web according to the present invention.

FIG. 4A is a top plan view of an absorbent article which has been insulted with a body fluid simulant and which depicts the stain resulting from the insult.

FIG. 4B is a top plan view of an absorbent article according to the present invention, with a fibrous nonwoven web according to the present invention, which has been insulted with a body fluid simulant and which depicts the stain resulting from the insult.

FIG. 4C is a top plan view of an absorbent article according to the present invention, with a fibrous nonwoven web according to the present invention, which has been insulted with a body fluid simulant and which depicts the stain resulting from the insult.

FIG. 4D is a side view photo of a prior art apertured nonwoven web.

FIG. 4E is a side view photo of a fibrous nonwoven web according to the present invention.

FIG. 5 is a plan view of a prior art apertured nonwoven web.

FIG. 6 is a top plan view of a fibrous nonwoven web according to the present invention.

FIG. 6A is a top plan view of a fibrous nonwoven web according to the present invention.

FIG. 6B is an enlarged top plan view of one of the embossed apertures from FIG. 6A.

FIG. 6C is an enlarged top plan view of one of the embossed apertures from FIG. 6A.

FIG. 7 is a side view photo of a fibrous nonwoven web according to the present invention.

FIG. 8 is a side view photo of a fibrous nonwoven laminate according to the present invention.

FIG. 9 is a plan view of the front surface of an aperturing and/or embossing design according to the present invention.

FIG. 10 is a plan view of the back surface of an aperturing and/or embossing design according to the present invention.

### DETAILED DESCRIPTION OF THE INVENTION

The present disclosure is directed to a fibrous nonwoven web with multiple arrays of embossing designs at least a portion of which terminate in apertures which facilitate fluid flow through the nonwoven web. As a result, the fibrous nonwoven web has a number of applications where fluid absorption and/or transfer are involved. This is particularly true with respect to absorbent articles, wipes and cleaning products. Also disclosed is the equipment and method for making such a nonwoven web material as well as end-use products employing such a material.

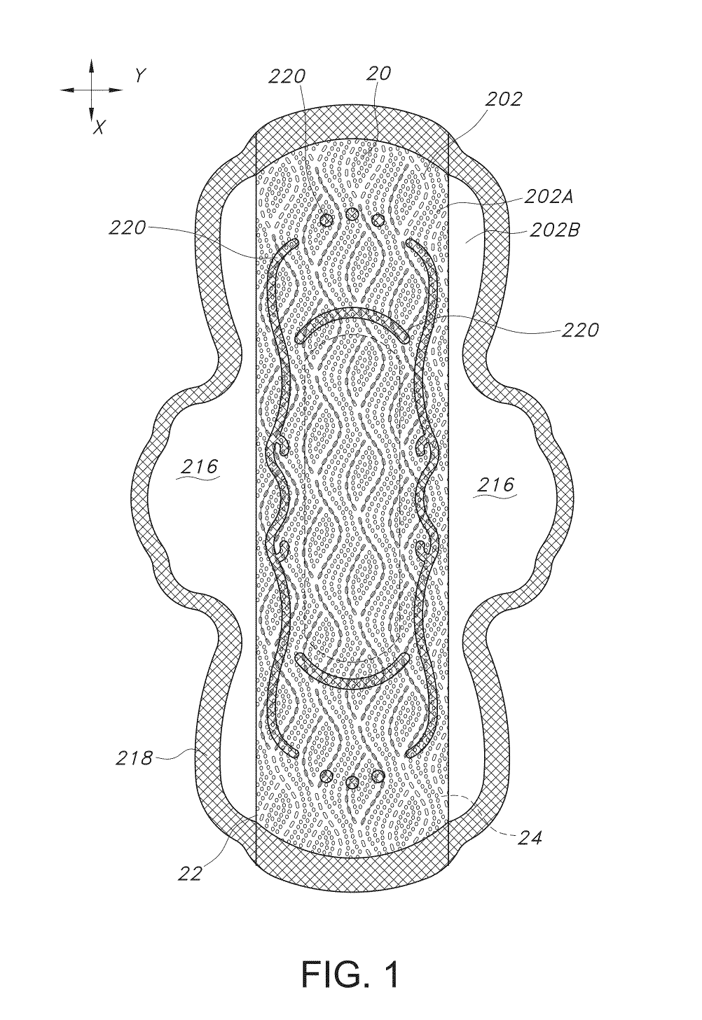

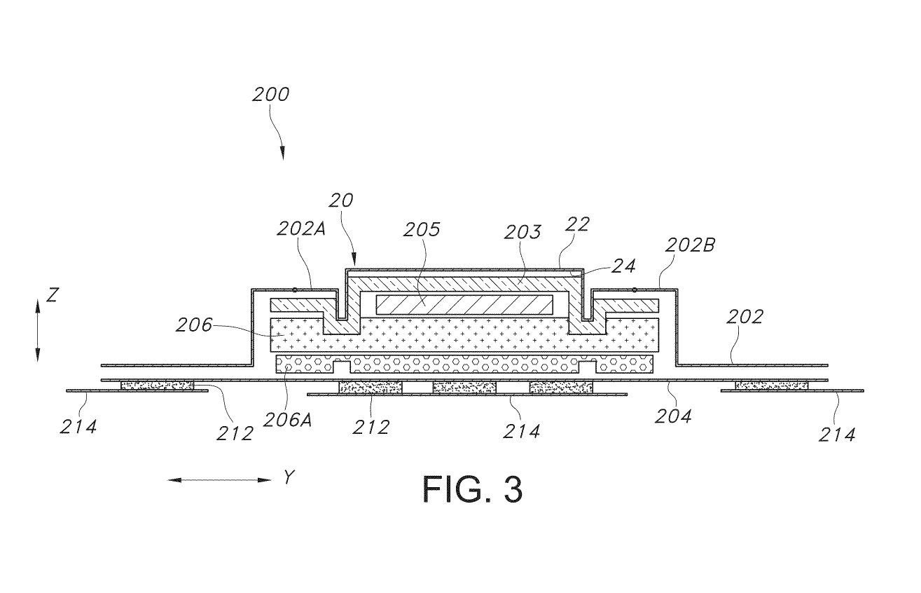

Turning to FIGS. 1 through 3 there is shown an absorbent article (200) also referred to as a personal care absorbent article. Examples of such absorbent articles include, but are not limited to, diapers, training pants, diaper pants, adult incontinence devices, feminine hygiene products, bandages and the like. Feminine hygiene products include a number of designs including, but not limited to, sanitary napkins and panty liners. The designs will vary depending on the capacity requirements regarding menstrual flow, the time of day or night they are being worn and the particular type of undergarment of the wearer. The particular absorbent article shown in FIGS. 1 through 3 is a sanitary napkin. For reference purposes, the absorbent article can be regarded as having a longitudinal or “X” axis, a transverse or “Y” axis and a vertical or “Z” axis. As shown, the absorbent article includes a topsheet or body side liner/layer (202), a backsheet or garment facing layer (204) and an absorbent core (206) disposed between the topsheet (202) and the backsheet (204). Typically the topsheet (202) and the backsheet (204) are sealed about their peripheries by some type of peripheral seal (218). Such seals can be accomplished through the use of, for example, adhesives, thermal bonding, ultrasonic bonding and combinations of the foregoing. In many applications where the absorbent article (200) is a feminine hygiene product such as a sanitary napkin, the absorbent article (200) can optionally include what are termed a pair of wings (216) positioned on the opposed lateral sides of the article (200) (in the direction of the Y axis) the purpose of which is to wrap around the undergarment of the wearer (not shown) to better hold the article (200) in place by way of a garment adhesive (212) which is covered prior to use by a release or peel strip (214). In addition, the exterior or garment facing side of the backsheet (204) can include garment adhesive (212) which is also covered by a peel strip (214). See FIG. 3.

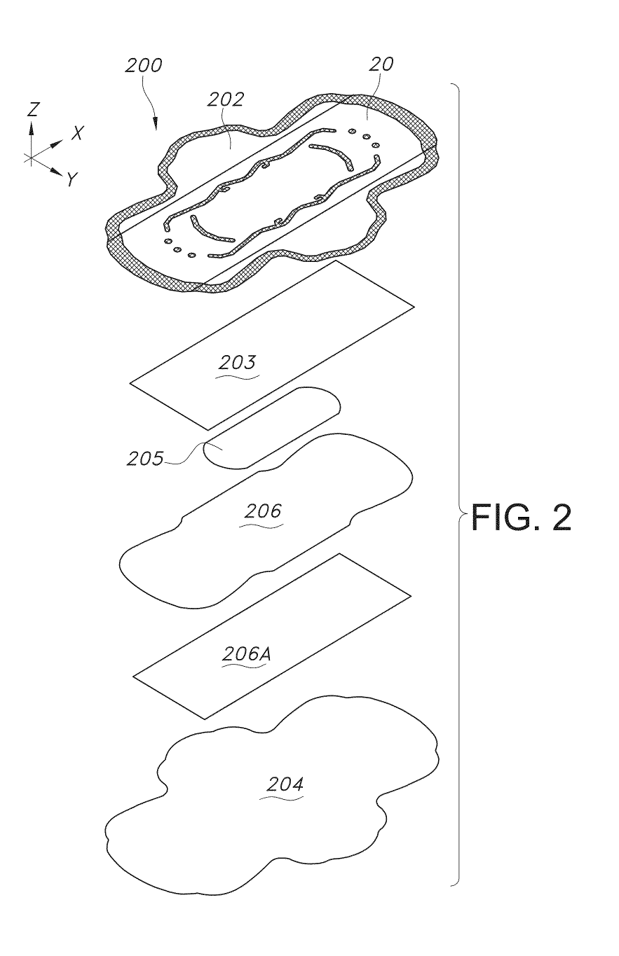

The foregoing description of an absorbent article (200) is relatively generic in design. If desired, additional optional layers may be added to the article (200) to enhance certain features of the article (200) and the overall performance. Referring to FIG. 2 which is an exploded view of the article (200) shown in FIG. 1, there is shown what is commonly referred to as a distribution or surge layer (203). The surge layer (203) is typically a layer that is designed to absorb sudden and oftentimes large gushes of body exudates such as urine, menses, blood, and loose or runny bowel movements. It is thus a fluid pervious material such as an apertured film or a fibrous nonwoven web. When the surge layer (203) is a fibrous nonwoven web, it is often the case that the web will have a more open structure than the topsheet (202) so as to more readily accept fluids received from the topsheet (202). It can be a mix of fibers both synthetic and natural and it can be made from a variety of nonwovens and forming techniques including, but not limited to, airlaid, coform, spunbond, meltblown, hydroentangled, and thermal and chemically bonded cared webs. Dimensionally, the surge layer (203) can be as wide and long as its adjacent layers or it can be shorter in either direction. Such surge layers (203), their make-up and there formation are known in the art. If desired, the surge layer (203) can be attached or unattached to the adjoining layers by such means as, for example, adhesives, thermal and point bonding, embossing, ultrasonic bonding and hydroentangling.

Directly below the surge layer (203) in the direction of the Z axis there can be placed a transfer layer (205). It too can be formed from the same materials and processes as the surge layer (203) though it will typically have some degree of difference in its functionality when compared to the other layers of the article (200). For example, if the transfer layer (205) is positioned between the surge layer (203) and the absorbent core (206), it may have functionality components of both adjoining layers (203 and 206). Thus, it may have the ability to absorb fluids faster than the absorbent core (206) and its retention capability may be higher than the surge layer (203) but less than the absorbent core (206). Dimensionally, the transfer layer (205) can be as wide and long as its adjacent layers or it can be shorter in either or both directions. Such transfer layers (205), their make-up and there formation are known in the art. If desired, the transfer layer (205) can be attached or unattached to the adjoining layers by such means as, for example, adhesives, thermal and point bonding, embossing, ultrasonic bonding and hydroentangling. The absorbent core (206) may be a single layer construction or it may be made of multiple layers in which case an optional and additional layer or layers (206A) may be employed.

The absorbent core (206) can be suitably constructed to be generally compressible, conformable, pliable, non-irritating to the wearer's skin and capable of absorbing and retaining liquid body exudates. The absorbent core (206) can be manufactured in a wide variety of sizes and shapes and from a wide variety of materials. The size and the absorbent capacity of the absorbent core (206) should be compatible with the size of the intended wearer and the liquid loading imparted by the intended use of the absorbent article (200). Additionally, the size and the absorbent capacity of the absorbent core (206) can be varied to accommodate wearers ranging from infants to adults. The absorbent core (206) may have a length and width that can be less than or equal to the length and width of the absorbent article (200).

In an embodiment, the absorbent core (206) can be composed of a web material of hydrophilic fibers, cellulosic fibers (e.g., wood pulp fibers), natural fibers, synthetic fibers, woven or nonwoven sheets, scrim netting or other stabilizing structures, superabsorbent material, binder materials, surfactants, selected hydrophobic and hydrophilic materials, pigments, lotions, odor control agents or the like, as well as combinations thereof. In an embodiment, the absorbent core (206) can be a matrix of cellulosic fluff and superabsorbent material.

In an embodiment, the absorbent core (206) may be constructed of a single layer of materials, or in the alternative, may be constructed of two or more layers of materials (206 and 206A). In an embodiment in which the absorbent core (206) has two layers, the absorbent core (206) can have a wearer facing layer (206) suitably composed of hydrophilic fibers and a garment facing layer (206A) suitably composed at least in part of a high absorbency material commonly known as superabsorbent material. In such an embodiment, the wearer facing layer of the absorbent core (206) can be suitably composed of cellulosic fluff, such as wood pulp fluff, and the garment facing layer of the absorbent core (206A) can be suitably composed of superabsorbent material, or a mixture of cellulosic fluff and superabsorbent material. As a result, the wearer facing layer (206) can have a lower absorbent capacity per unit weight than the garment facing layer (206A). The wearer facing layer (206) may alternatively be composed of a mixture of hydrophilic fibers and superabsorbent material, though it is generally desirable that the concentration of superabsorbent material present in the wearer facing layer (206) be lower than the concentration of superabsorbent material present in the garment facing layer (206A) so that the wearer facing layer (206) can have a lower absorbent capacity per unit weight than the garment facing layer (206A). It is also contemplated that, in an embodiment, the garment facing layer (206A) may be composed solely of superabsorbent material without departing from the scope of this disclosure. It is also contemplated that, in an embodiment, each of the layers, the wearer facing (206) and garment facing (206A) layers, can have a superabsorbent material such that the absorbent capacities of the two superabsorbent materials can be different and can provide the overall absorbent core (206) with a lower absorbent capacity in the wearer facing layer (206) than in the garment facing layer (206A).

Various types of wettable, hydrophilic fibers can be used in any of the layers of the absorbent core (206). Examples of suitable fibers include natural fibers, cellulosic fibers, synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester or polyamide fibers, or composed of nonwettable thermoplastic polymers, such as polyolefin fibers which have been hydrophilized by suitable means. The fibers may be hydrophilized, for example, by treatment with a surfactant, treatment with silica, treatment with a material which has a suitable hydrophilic moiety and is not readily removed from the fiber, or by sheathing the nonwettable, hydrophobic fiber with a hydrophilic polymer during or after formation of the fiber. For example, one suitable type of fiber is a wood pulp that is a bleached, highly absorbent sulfate wood pulp containing primarily soft wood fibers. However, the wood pulp can be exchanged with other fiber materials, such as synthetic, polymeric, or meltblown fibers or with a combination of meltblown and natural fibers. In an embodiment, the cellulosic fluff can include a blend of wood pulp fluff.

The absorbent core (206) can be formed with a dry-forming technique, an air-forming technique, a wet-forming technique, a foam-forming technique, or the like, as well as combinations thereof. A coform nonwoven material may also be employed. Methods and apparatus for carrying out such techniques are known in the art. Suitable superabsorbent materials can be selected from natural, synthetic, and modified natural polymers and materials. The superabsorbent materials can be inorganic materials, such as silica gels, or organic compounds, such as cross-linked polymers. Cross-linking may be covalent, ionic, Van der Waals, or hydrogen bonding. Typically, a superabsorbent material can be capable of absorbing at least about ten times its weight in liquid. In an embodiment, the superabsorbent material can absorb more than twenty-four times its weight in liquid. Examples of superabsorbent materials include polyacrylamides, polyvinyl alcohol, ethylene maleic anhydride copolymers, polyvinyl ethers, hydroxypropyl cellulose, carboxymal methyl cellulose, polyvinylmorpholinone, polymers and copolymers of vinyl sulfonic acid, polyacrylates, polyacrylamides, polyvinyl pyrrolidone, and the like. Additional polymers suitable for superabsorbent material include hydrolyzed, acrylonitrile grafted starch, acrylic acid grafted starch, polyacrylates and isobutylene maleic anhydride copolymers and mixtures thereof. The superabsorbent material may be in the form of discrete particles. The discrete particles can be of any desired shape, for example, spiral or semi-spiral, cubic, rod-like, polyhedral, etc. Shapes such as needles, flakes, and fibers are also contemplated for use herein. Conglomerates of particles of superabsorbent materials may also be used in the absorbent core (206).

The absorbent core (206) can be superposed over the backsheet (204) and can be bonded to the backsheet (204), such as by being bonded thereto with an adhesive. However, it is to be understood that the absorbent core (206) may be in contact with, and not bonded with, the backsheet (204). In an embodiment, a layer, such as but not limited to, a core wrap, can be used to partially or completely envelope the absorbent core (206) layer or layers.

If desired, the absorbent core (206) can optionally be partially or wholly enveloped by what is termed a core wrap (not shown in the drawings), the primary function of which is to contain the materials forming the absorbent core (206), especially when materials such as superabsorbent particles are being used to absorb body exudates. The core wrap can be in contact with and if desired, bonded to the absorbent core (206). Bonding of the core wrap to the absorbent core (206) can occur via any means such as, but not limited to, adhesives. The core wrap may be composed of separate sheets of material which can be utilized to partially or fully encompass the absorbent core (206) and which can be sealed together using a sealing means such as an ultrasonic bonder or other thermochemical bonding means or by the use of an adhesive. The core wrap can include, but is not limited to, natural and synthetic fibers and can be made to be more or less hydrophilic through the use of surfactant chemicals such as are known in the construction of such materials. Suitable materials include, but are not limited to, tissue wraps and meltblown fibrous nonwoven webs.

The backsheet (204) can be breathable and/or liquid impermeable. The backsheet (204) can be elastic, stretchable or non-stretchable. The backsheet (204) may be constructed of a single layer, multiple layers, laminates, spunbond fabrics, films, meltblown fabrics, elastic netting, microporous webs, bonded-carded webs or foams provided by elastomeric or polymeric materials. For example, the backsheet (204) can be constructed of a microporous polymeric film, such as polyethylene or polypropylene or it can be such a film laminated to a fibrous nonwoven web such as a layer of spunbond or a bonded carded web made from, for example, polyolefins such as polyethylene or polypropylene. In an embodiment, the backsheet (204) may be a two layer construction (not shown), including an outer layer material and an inner layer material which can be bonded together such as by a laminate adhesive. Suitable laminate adhesives can be applied continuously or intermittently as beads, a spray, parallel swirls, or the like. In addition, the inner layer can be bonded to the outer layer utilizing ultrasonic bonds, thermal bonds, pressure bonds, or the like. The outer layer of the backsheet can be any suitable material and may be one that provides a generally cloth-like texture or appearance to the wearer. An example of such material can be a polypropylene bonded-carded web. Another example of material suitable for use as an outer layer of a backsheet (204) can be a spunbond polypropylene non-woven web.

The liquid impermeable inner layer of the backsheet (204) (or the liquid impermeable backsheet (204) where the backsheet (204) is of a single-layer construction) can be either vapor permeable (i.e., “breathable”) or vapor impermeable. The liquid impermeable inner layer (or the liquid impermeable backsheet (204) where the backsheet (204) is of a single-layer construction) may be manufactured from a thin plastic film, although other liquid impermeable materials may also be used. The liquid impermeable inner layer (or the liquid impermeable backsheet (204) where the backsheet (204) is of a single-layer construction) can inhibit liquid body exudates from leaking out of the absorbent article (200) and wetting articles, such as bed sheets and clothing, as well as the wearer and caregiver.

Where the backsheet (204) is of a single layer construction, it can be embossed and/or matte finished to provide a more cloth-like texture or appearance. The backsheet (204) can permit vapors to escape from the absorbent article (200) while preventing liquids from passing through. A suitable liquid impermeable, vapor permeable material can be composed of a microporous polymer film or a non-woven material which has been coated or otherwise treated to impart a desired level of liquid impermeability.

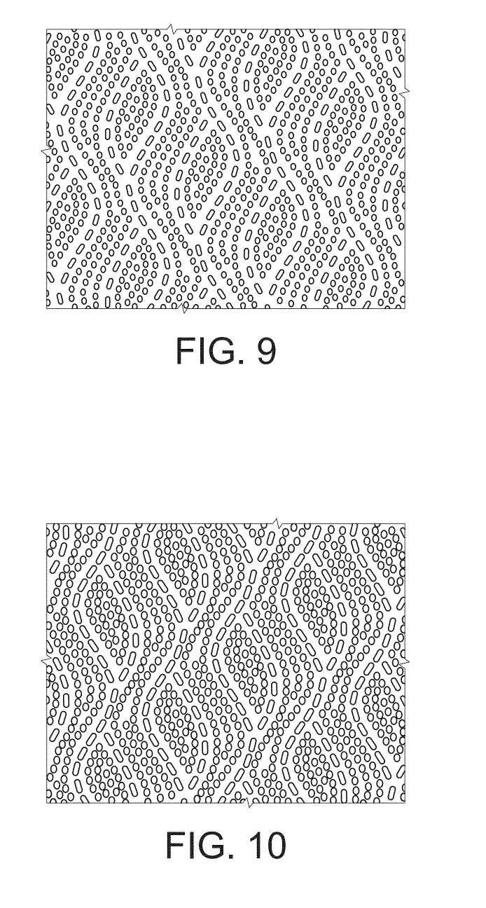

The topsheet (202) of the absorbent article (200) may be made from a variety of materials including fibrous nonwoven webs and apertured films, both of which may employ the aperture-embossed pattern shown from the front in FIG. 9 and the back in FIG. 10 of the drawings. The topsheet (202) in FIG. 1 is what is often referred to as a dual layer cover as it has a central portion or strip (202A) straddled by two opposed lateral strips of material (202B) which may be the same or different from the central portion (202A). Such a two layer or dual layer cover configuration is described for example, in U.S. Pat. No. 5,961,505 to Coe, U.S. Pat. No. 5,415,640 to Kirby and U.S. Pat. No. 6,117,523 to Sugahara, each of which is hereby incorporated by reference in its entirety. It is also contemplated that such two layer topsheet materials may additionally include elastic components (not shown) along their side edges to lift up portions of the side materials during use, thereby forming physical barriers or cupping features on the product so as to fit more closely to the body of a user.

While the aperture-embossed pattern of FIGS. 9 and 10 may be used for the central portion (202A) of the topsheet (202), it should be appreciated that this pattern can be used across the entire topsheet (202) as well as other layers and components of the absorbent article (200). Further, it may be used in connection with both film and fibrous nonwoven web materials as well as combinations of the foregoing.

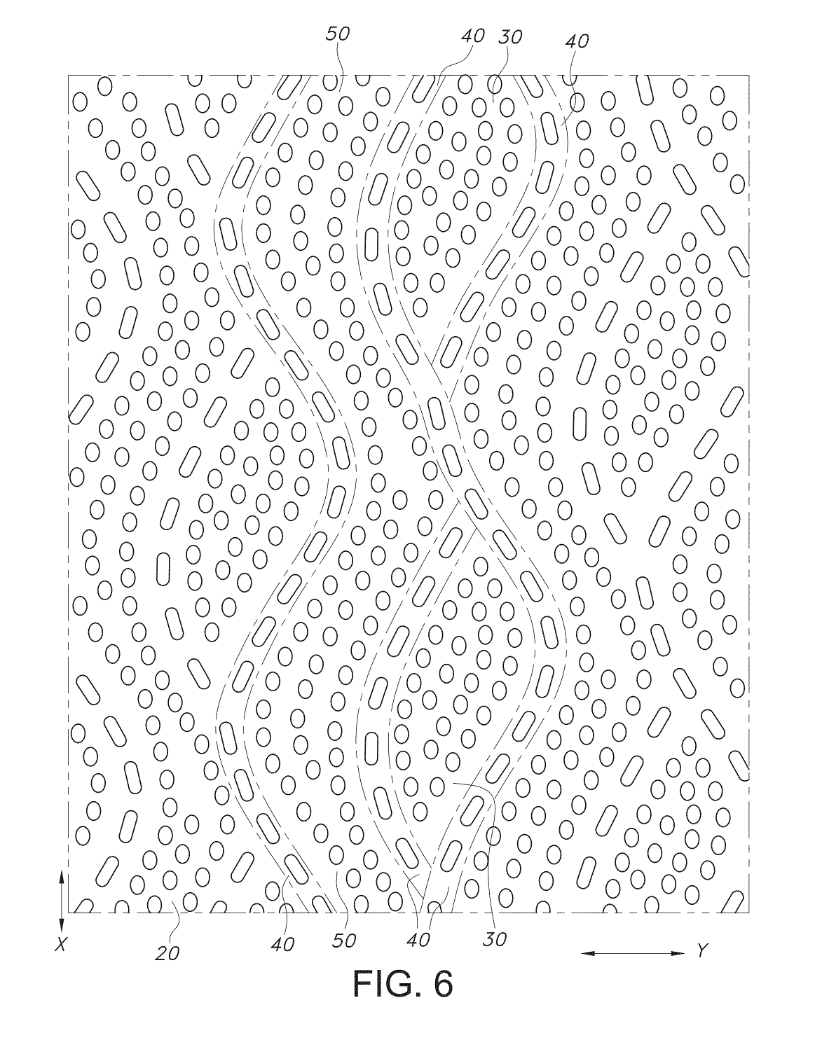

In the embodiment shown in FIG. 1, 4B and 4C, the central portion (202A) is formed from a fibrous nonwoven web (20) such as is shown in FIGS. 6 and 6A. Turning to the combination of FIGS. 1, 4E, 6, 6A, 6B and 6C there is shown a fibrous nonwoven web (20) having a top surface (22) and a bottom surface (24). The web (20) is provided with a plurality of varying arrays of apertures formed in the bottom of depressions formed from the top surface (22) down into the thickness of the web (20). In this regard it should be noted that with respect to fibrous nonwoven webs that “apertures” should be distinguished from the naturally occurring interstitial spaces existing between the fibers or filaments forming the web. By “array” it is generally meant that there are visually distinct and separate sections or areas of apertures and/or depressions that form a shape which can be detected when viewed with the naked eye. For example, in FIGS. 1 and 6 there is shown a first array (30) of first apertures (32) that are formed in a plurality of first depressions (34). While only an example and not restricting the scope of the description and claims, the first array (30) visually looks like a leaf. The web (20) also has a second array (40) of second apertures (42) that are formed in a plurality of second depressions (44). While only an example and not restricting the scope of the description and claims, the second array (40) visually looks like a sinusoidal wave. In the same fashion, the web (20) is provided with a third array (50) of third apertures (52) that are formed in a plurality of third depressions (54) that also look like a sinusoidal wave.

The fibrous nonwoven web (20) of the present invention can be prepared from non-continuous fibers (such as staple fibers), continuous filaments (such as meltblown and spunbond fibers) as well as combinations thereof. Other shorter fibers, such as pulp fibers can also be used. The production methods that may be employed include, but are not limited to, dry staple processes such as carded web techniques (hot thru-air bonded carded web and thermal bonded carded web techniques), meltblowing processes, spunbond processes, hydroentangling process and generally any wet or dry forming processes as well as other fiber extrusion processes. Through air bonded carded webs (TABCW) are particularly well-suitable as they can form bulky webs which include large quantities of vacant, interstitial spaces between the fibers to create a low-density web. As a result, when the depressions and apertures are formed, the resultant web has a more three-dimensional look and feel due to the increased depth and fluids imparted to the web can be quickly absorbed and passed to subjacent layers. The fibers forming the webs can be made from both natural and synthetic materials. Suitable polymers for fiber formation include, but are not limited to, thermoplastic polymeric materials such as polyolefins including, for example, polyethylene and polypropylene as well as polyamides, polyesters and the like. The fibers can be solid or hollow core fibers, shaped fibers, single polymer/component fibers as well as multi-constituent fibers such as bicomponent fibers. The same materials, fibers and webs and web-forming techniques used to form the web (20) and topsheet (202) can be employed for the subjacent layers (203), (205) and vice versa.

Turning to FIG. 6, it can be seen that the first array (30) is at least partially surrounded by the second array (40) and that the second array (40) separates the first array (30) from the third array (50). Additionally, the second array (40) can completely surround the first array (30) and/or it can partially or completely surround the third (50) or other additional arrays if so desired. The same is true with either or both of the first (30) and third (50) and additional arrays.

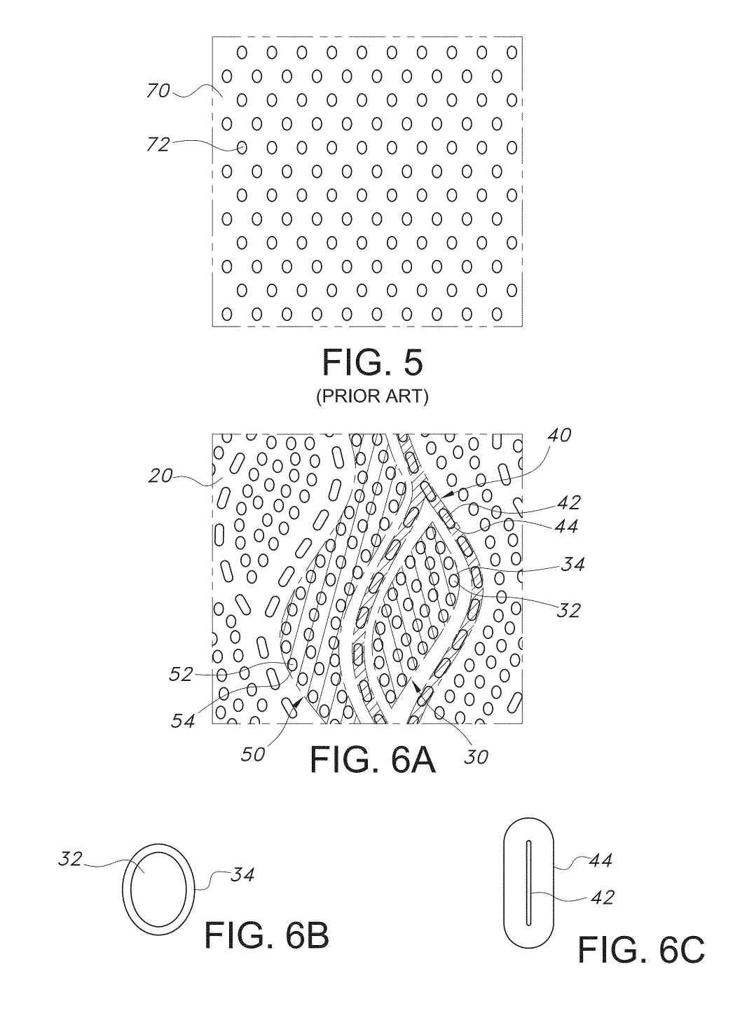

FIG. 5 shows a conventional and commercially available fibrous nonwoven web (70) with a standard, uniform aperturing pattern of apertures (72). It has been found that the providing the material with non-linear and varied patterns of perforations and depressions provides the consumer with a more visually appealing material that is preferred over linear, uniform patterns. In addition, as shown in more detail below, such non-linear patterns provide better fluid transfer for liquids and semi-solids such as body exudates including menses, blood, urine, and low viscosity feces. Further, from a material handling and processing standpoint, it was discovered that the machine direction (MD) and cross-machine (CD) tensile strength of the materials were improved over uniform aperturing and embossing patterns resulting in more efficient manufacturing and conversion.

In one embodiment, to form the visually distinct pattern shown in FIGS. 1 and 6, it is desirable that the first density of the first array (30) of first apertures (32) be different from and alternately greater that the second density of the second array (40) of second apertures (42) and that the second density of the second array (40) of second apertures (42) be different from and alternatively less than the third density of the third array (50) of third apertures (52). By “density” it is meant the number of apertures and/or depressions per unit area in a selected area of a particular array.

As described above, the aperturing and embossing pattern shown in FIGS. 1, 6 and 6A has alternating patterns of a leaf-type structure (array (30)) separated by two sinusoidal patterns (arrays (40) and (50)). This pattern can be altered in numerous ways without departing from the spirit and scope of the present invention. For example, the wave length of the sinusoidal patterns can be varied as to the values for the wave peak amplitude, the wave length and the wave peak-to-peak (peak to adjacent trough) amplitude measurements. Also, the juxtaposition of adjacent sinusoidal wave patters can be varied. They can be skewed or out-of-phase such as is shown in FIGS. 1, 6 and 6A. For example, the peak-to-peak alignment of adjacent sine waves in the referenced drawings is skewed at an angle of approximately 40 degrees. This degree of skewing can be varied. Conversely, adjacent sine waves can be aligned and thus in phase as to their peaks and troughs.

The wave length of the sine waves used in the arrays (40) and/or (50) can range from about 20 to about 50 millimeters and alternatively from about 30 to about 40 millimeters though wavelengths outside this range are also possible depending on the particular end use. The peak-to-peak wave amplitude can range from about 3 to about 30 millimeters and alternatively from about 5 to about 15 millimeters though amplitudes outside this range are also possible depending on the particular end use.

The number of repeating sinusoidal patterns in a particular array can also be varied. For example, in FIGS. 1, 6 an 6A the number of patterns is two in some areas of the third array (50) and three in other areas of the array (50). This can be varied to increase or decrease the number of repeating patterns. In array (40) there is shown a single sinusoidal pattern but this can be increased to a plurality of adjacent sinusoidal patterns. Further the designs of the second array (40) and the third array (50) can be reversed.

Again referring to FIGS. 6 and 6A is can be seen that there is a repeating pattern to the arrays (30), (40) and (50) within the overall pattern shown in these Figures. Sequentially, the arrays can repeat themselves vertically and/or horizontally in the overall pattern. Also, it can be seen that the arrays can partially or completely surround one another. For example, the array (40) is completely surrounding the array (30) and the array (40) also separates array (30) from array (50).

The spacing between arrays ((30), (40) and (50)) can be varied. If desired, areas with no embossing and/or aperturing (also sometimes referred to as land area) can be interposed between arrays or within arrays.

The size and shape of the leaf-type pattern in the first array (30) can be varied as to design and shape. Generally the length of the leaf-type shapes will range from about 5 to about 40 millimeters, alternatively from about 10 to about 25 millimeters. Generally, the width of the leaf-type shapes will range from about 3 to about 25 millimeters, alternatively between about 5 and about 15 millimeters. Other sizes can be used depending upon the particular end use of the material.

It should be understood that the fibrous nonwoven web (20) and the individual arrays are not limited to the specific designs shown herein. Each of the arrays can take on a variety of shapes and sizes and fulfill the requirements of the present invention. While the aperturing and embossing pattern shown in FIGS. 1, 6 and 6A is comprised of sinusoidal waves and leaf-like patterns, other patterns and combinations of patterns are also possible. Any of the arrays can be made more or less irregular, larger or smaller. Further, while at least three arrays are preferred, higher numbers of arrays may also be employed including, for example, four, five, six and more.

Turning to FIGS. 6A, 6B and 6C, the first array (30), second array (40) and third array (50) each have a plurality of depressions formed by the embossing portion of the process described below. At least a portion and preferably all of the depressions terminate with an aperture located in the bottom of the depressions which are also formed as a result of the process described below. An enlarged top plan view of a first depression (34) with a first aperture (32) is shown in FIG. 6B. An enlarged top plan view of a second depression (44) with a second aperture (42) is shown in FIG. 6C. Comparing the two, it can be seen that the degree of open area due to the first aperture (32) in the first depression (34) is larger than the degree of open area due to the second aperture (42) in the second depression (44). This is due to the degree of taper between the beginning of the depression starting in the top surface (22) of the fibrous nonwoven web (20) versus the taper and the termination point of the depression (34) which defines the aperture (32). The sidewalls of the first depressions (34) thus have steeper sidewalls than the sidewalls of the second depressions (44). The degree of tapering can be varied between depressions in one array versus another and within a particular array.

Spacing between apertures/depressions within an array can be varied in any direction. Generally, edge-to-edge spacing between depressions as measured at the top surface (22) will range between about 1.0 and about 4.0 millimeters, alternatively between about 1.0 and about 3.0 millimeter, and still further between about 2.0 and about 4.0 millimeters.

The plane of the bottom surface (24) of the fibrous nonwoven web (20) will be defined, at least in part, by the termination point of the depressions with their open apertured ends. Depending on the degree of taper and the depth of the depressions, some of the depressions, with or without apertures, may terminate in and define the plane of the bottom surface (24) of the fibrous nonwoven web (20) while other depressions may terminate at a location intermediate the top surface (22) and the bottom surface (24). In some instances it may be the case that the depressions and apertures within one array partially or as a whole are deeper than the depressions and apertures in another array. For example, with respect to the aperturing and embossing design shown in FIGS. 1, 6 and 6A, the depth or vertical height (relative to the Z axis) of the second depressions (44) and second apertures (42) in the second array (40) are greater than the depressions and apertures in the first array (30) and third array (50). Thus, when the fibrous nonwoven web (20) bottom surface (24) is placed adjacent and preferably in contact with a subjacent layer (such as surge layer (203)), the second depressions (44) will, in essence, act as “legs” to support the web (20) on top of the subjacent layer, thereby forming air pockets (first array (30)) and air channels (third array (50)) below the bottom surface (24) of the web (20). Another advantage of such a design is the increased bulk and cushion-like feel that is imparted to the web (20) and the resultant article (200) without an increase in basis weight.

### EXAMPLES

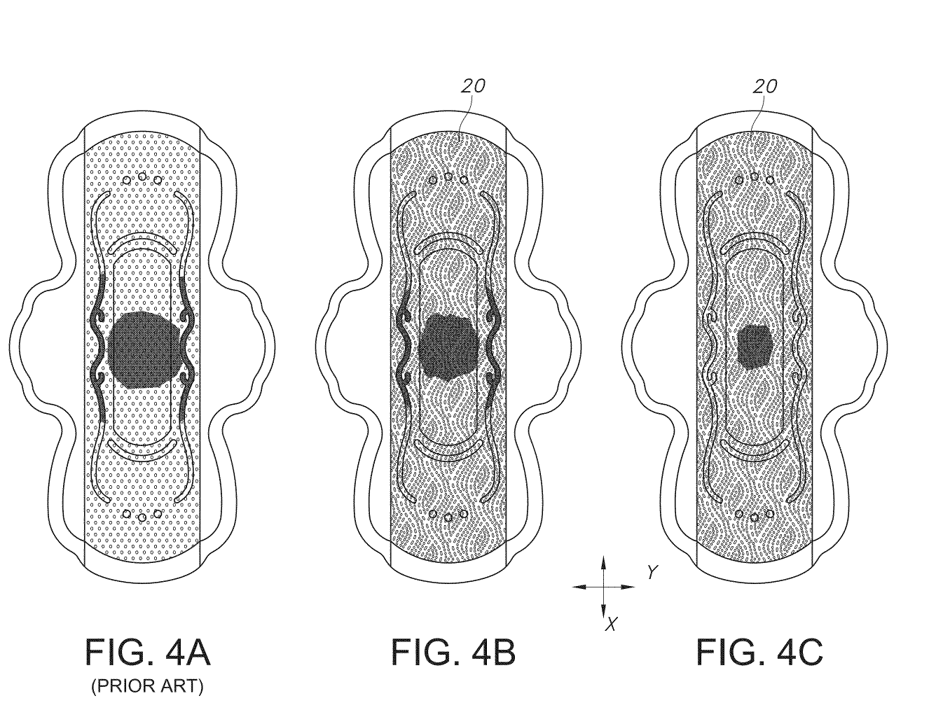

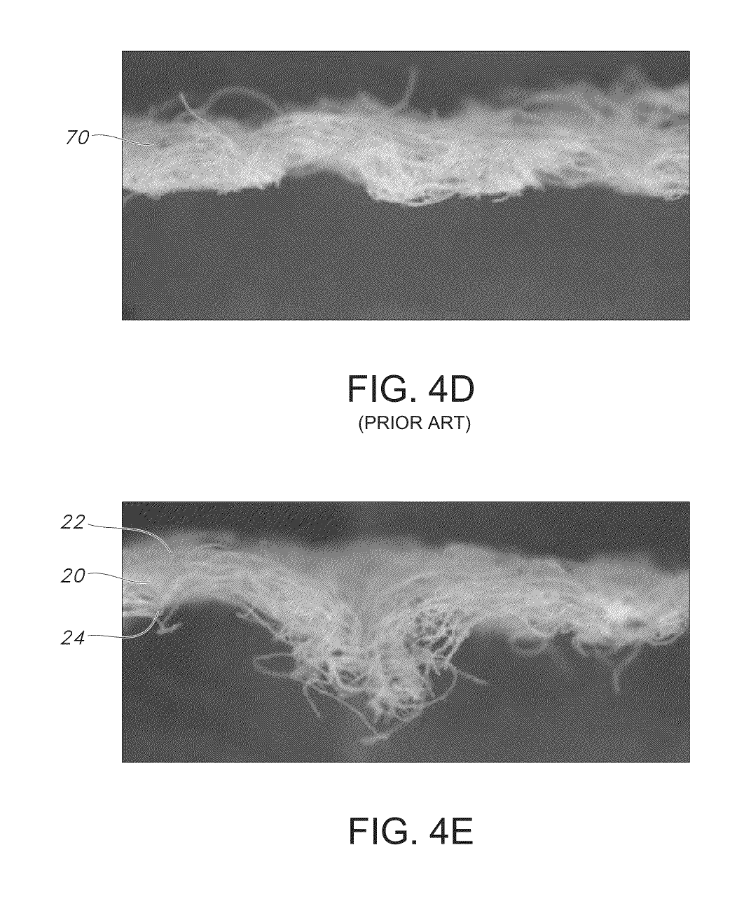

To demonstrate the improved fluid handling and stain masking characteristics of the fibrous nonwoven web (20) described herein, three absorbent articles (200) were made with identical constructions except for the topsheet (202). The absorbent articles (200) were in the form of sanitary napkins similar in design to those shown in FIGS. 1 through 3 of the drawings. Representations of the three sanitary napkins are shown in FIGS. 4A, 4B and 4C of the drawings. The sanitary napkin in FIG. 4A was adapted from a commercially available sanitary napkin available from Yuhan-Kimberly, Ltd. of Seoul, South Korea and sold under the trade designation KOTEX® GOODFEEL™. The sanitary napkins of FIGS. 4B and 4C used the same chassis as the sanitary napkin in FIG. 4A but utilized fibrous nonwoven webs (20) according to the present invention. Describing the chassis in a bottom up configuration in the direction of the Z axis, the backsheet (204) was a polyethylene film. Continuing in the direction of the Z axis, the absorbent core (206) was a two layer structure having a lower layer (206A) adjacent the backsheet (204) and an upper layer (206) on the top or body facing side of the lower layer (206A). The lower layer (206A) was a superabsorbent and pulp fluff-containing sheet wrapped with a tissue core wrap. The upper layer (206) of the absorbent core was a layer of airlaid material made from a blend of pulp fibers and staple fibers.

Positioned on top of the airlaid upper layer (206) was a smaller two layer TABCW transfer layer (205) in which the upper layer was printed with a purple color so as to be visible through the topsheet (202). Positioned between the purple transfer layer (205) and the topsheet (202) was a surge layer (203) which was also a TABCW structure. The addition of color was different from the commercially available product. Having one of the subjacent layers be darker in color from the layers above served the function of improving the overall stain masking function of the absorbent article (200) and this was evident in all three of the example products shown in FIGS. 4A, 4B and 4C. By “darker” in color, independent of the use of this term in the present example, it is meant that the color of the layer in question is sufficiently different from the other layers in the article and in particular the layers above it (towards the body-contacting side of the article along the Z axis) such that the darker color can be visually observed by the naked eye through the topsheet when the article is held at a point in the range of 30 to 90 centimeters from the human eye.

The topsheet on the product shown in FIG. 4A had a central portion or strip (202A) of TABCW material laterally straddled by and attached on its opposite lateral sides to layers (202B) of hydrophobic TABCW material. The central strip (202A) of TABCW had a basis weight of 24 gsm and utilized 1.8 denier, 38 millimeter long, polyethylene sheath/polypropylene core bicomponent staple fibers. The central portion (202A) of the topsheet (202) was apertured with an 18 pin uniform aperturing pattern as shown In FIGS. 4A and 5. The apertures were oval in shape with a major axis in the range of 0.9 to 1.5 millimeters and a minor axis in the range of 0.7 to 1.1 millimeters. The pin aperturing gave a hole density of approximately 18 apertures per square centimeter. A photo of this material in cross-section is shown in FIG. 4D of the drawings. The central strip (202A) of apertured nonwoven was attached to lateral side strips (202B) of 30 gsm TABCW utilizing hydrophobic polyethylene sheath/polypropylene core bicomponent staple fibers. The length and width of the central strip (202A) of nonwoven material will vary depending on the size of the product but was approximately 24 centimeters long and approximately 8 centimeters wide for the samples described herein.

The overall product was embossed with channels (220) as shown in FIGS. 1 and 4A and peripherally sealed (218) using heat embossing and adhesive. The topsheet utilized for the central portion (202A) had a thickness of 0.76 millimeters. See FIG. 4A.

The second example (FIG. 4B) had the same chassis as the first example (FIG. 4A) but the central portion (202A) utilized a fibrous nonwoven web (20) according to the present invention. The fibrous nonwoven web (20) for the central portion (202A) was a two layer structure TABCW as viewed along the Z axis having an overall basis weight of 24 gsm. The top or body-contacting layer of the central portion (202A) was hydrophilic in nature and comprised a 12 gsm through-air bonded carded nonwoven web utilizing polyethylene sheath/polypropylene core, 1.5 denier, 38 millimeter long bicomponent staple fibers. The bottom layer comprised a 12 gsm TABCW nonwoven web of polyethylene sheath/polyester core, 2.0 denier, 38 millimeter long bicomponent staple fibers. The combined, two-layer web (202A) was subjected to hot through air bonding. The 24 gsm web was embossed and apertured with the pattern shown in FIGS. 1 and 6. It too was attached to the same type of side strips (202B) of topsheet material as the first example described above and was attached to the chassis in the same matter. Also, it had the same embossed channels (218) as the first example. The topsheet utilized for the central portion (202A) had the same length and width dimensions as the first example and had a thickness of 0.90 millimeters as measured while under a pressure of 0.56 grams per square centimeter. See FIG. 4B.

The third example (FIG. 4C) had the same chassis as the first example (FIG. 4A) but the central portion (202A) also utilized a fibrous nonwoven web (20) according to the present invention. The fibrous nonwoven web (20) for the central portion (202A) was a two layer structure with an overall basis weight of 24 gsm. The top or body-contacting layer was more hydrophobic in nature than the second example and comprised a 12 gsm TABCW utilizing polyethylene sheath/polypropylene core, 1.5 denier, 38 millimeter long hydrophobic bicomponent staple fibers. The bottom layer comprised a 12 gsm TABCW of polyethylene sheath/polyester core, 2.0 denier, 38 millimeter long bicomponent staple fibers. The combined, two-layer web (202A) was subjected to hot through air bonding. The 24 gsm web was embossed and apertured with the pattern shown in FIGS. 1 and 6. It too was attached to the same type of side strips (202B) of topsheet material as the first example described above and was attached to the chassis in the same matter. Also, it had the same embossed channels (218) as the first example. The topsheet utilized for the central portion (202A) had the same length and width dimensions as the first example and had a thickness of 0.90 millimeters as measured while under a pressure of 0.56 grams per square centimeter. See FIG. 4C.

### Testing

To demonstrate the difference in the fluid handling properties of the three examples shown in FIGS. 4A, 4B and 4C, each product was laid flat on a work surface and was insulted with 6 milliliters of synthetic menstrual fluid over the center point of the topsheet at a rate of approximately 0.2 milliliters per second at a distance of approximately 1.5 centimeters above the top surface of the sanitary napkins. The fluid was allowed to absorb for approximately 3 minutes with no pressure being applied to the products at which point photos were taken of each of the products. Synthetic menstrual fluids and their formulations can be found, for example, in U.S. Pat. Nos. 5,883,231 and 6,932,929 as well as the publication by D, Guralski, Candee Krautkramer, Brian Lin, Jack Lindon, Teuta Elshani, Aneshia Ridenhour, entitled “A Biological Menses Simulant Using a “Batch” Homogenization Process”, and published as Document IPCOM000198395D at ip.com, 6 Aug. 2010, each of which are hereby incorporated by reference in their entirety.

The results of the fluid depositions are shown schematically in FIGS. 4A, 4B and 4C. As can be seen, the stain size in the first example (FIG. 4A) (the uniform apertured topsheet) was the largest of all three. The second sample (FIG. 4B) was noticeably smaller in size demonstrating that there was greater penetration of the deposited fluid which resulted in a drier topsheet which in turn is more comfortable to the wearer. In addition, the visual effect of the smaller stain is to improve the confidence level of the wearer that the product is working well and providing adequate protection. Turning to FIG. 4C, it can be seen that by increasing the hydrophobicity of the topsheet, an even higher penetration of the deposited fluid was achieved thereby further improving the described benefits of the second sample shown in FIG. 4B. The stain size in FIG. 4B was reduced to at least two-thirds the size of the sample of FIG. 4A and the stain size of the third sample in FIG. 4C was well less than half the size of the stain in FIG. 4A. Thus, it can be seen that the apertured topsheet (202) of the present invention was a significant improvement over the existing topsheet design as to fluid absorption. In addition to a reduced stain size on the topsheet, the stain masking was also improved due to the use of the purple transfer layer (205) which served to further block the visual observance of the absorbed menstrual fluid in the lower layers of the product.

The absorbent article examples described above were evaluated with respect to one another and the products employing the fibrous nonwoven web (20) according to the present invention (FIGS. 4B and 4C) were preferred on a number of bases. Attributes such as quickly absorbing menstrual fluid, feeling drier, being more breathable and masking menstrual stains were regarded as being preferred over the product with the conventional topsheet (FIG. 4A). The topsheet (20) used in the products of FIGS. 4B and 4C was also preferred as having a more three-dimensional look and feel.

Based upon the foregoing information and testing it was demonstrated that improved fluid intake and stain masking as well as other comfort benefits could be achieved when a fibrous nonwoven web according to the present invention was used as a topsheet in conjunction with a personal care absorbent article such as, for example, a feminine hygiene product including a sanitary napkin. It is believed that the improved performance was based on creating a topsheet with three or more different arrays of apertures with varying densities. In addition, by providing embossed depressions with differing vertical heights as measured along the vertical or Z axis (the direction normal to the plane of the product when laid flat), increases in fluid handling were achieved. These differences in vertical height were the result of the size and the depth of the depressions that were created by the embossing and aperturing pins used to form the combination depressions and apertures in in the various arrays of the fibrous nonwoven web (20).

As can be seen in FIGS. 1 and 6, the density of the apertures and accompanying depressions can vary from array to array. Thus the first density of the first array (30) of first apertures could be greater than, equal to or less than either or both of the second array (40) of second apertures (42) and the third array (50) of third apertures (52). The same is true of the second array (40) of second apertures (42) relative to either or both of the first array (30) of first apertures (32) and the third array (50) of third apertures (52). Lastly, the same is true of the third array (50) of third apertures (52) relative to either or both of the first array (30) of first apertures (32) and the second array (40) of second apertures (42). By “greater than” it is meant that the parameter in question is at least 10 percent greater than the parameter to which it is being compared. By “less than” it is meant that the parameter in question is at least 10 percent less than the parameter to which it is being compared. When the term “different” or “difference” or variances of this term are being used herein as in the case of, for example, depression depth, depression density, depression open area, aperture density, aperture open area, it is meant that the parameter in question is at least 10 percent different (higher or lower in value) than the parameter to which it is being compared. When the same term is used in the context of “different” shape, it is meant that two or more shapes can be distinguished from one another when visually observed by the naked eye when the article is held at a point in the range of 30 to 90 centimeters from the human eye.

Generally, the total open area of the first apertures (32) per unit area of the first array (30) of the nonwoven web (20) should be between about 2 and about 20 percent, alternatively between about 3 and about 10 percent and still further between about 4 and about 6 percent. Generally and independent of the first array (30) the total open area of the second apertures (42) per unit area of the second array (40) of the nonwoven web (20) should be between about 1 and about 10 percent, alternatively between about 1 and about 4 percent and still further between about 2 and about 3 percent. Generally and independent of the first array (30) and the second array (40), the total open area of the third apertures (52) per unit area of the third array (50) of the nonwoven web (20) should be between about 2 and about 20 percent, alternatively between about 6 and about 11 percent and still further between about 8 and about 9 percent. Because the apertures themselves reside below the plane of the top surface (22) of the fibrous nonwoven web (20) which forms the topsheet (202), it should be noted that these open areas are for the apertures ((32), (42), (52)) themselves at the bottom of the depressions ((34), (44), (54)) as compared to the total surface area of the array ((30), (40), (50)) being used to calculate the percent open area including the area of the openings in the top surface (22) formed by the depressions ((34),(44),(54)) formed within the particular array ((30), (40), (50)) and the land area between the depressions. Thus, the percent open area would be the total area of the collective apertures divided by the total area of the portion of the array being measured with the quotient being multiplied by 100 to yield the percentage. Note that within an array if any portion of the area or an array chosen meets these parameters, the array as a whole is considered to meet these parameters.

Generally, the total open area of the first depressions (34) per unit area of the first array (30) of the nonwoven web (20) should be between about 3 and about 25 percent, alternatively between about 5 and about 13 percent and still further between about 8 and about 9.5 percent. Generally and independent of the first array (30) the total open area of the second depressions (44) per unit area of the second array (40) of the nonwoven web (20) should be between about 2 and about 30 percent, alternatively between about 5 and about 20 percent and still further between about 10.5 and about 12.5 percent. Generally and independent of the first array (30) and the second array (40), the total open area of the third depressions (54) per unit area of the third array (50) of the nonwoven web (20) should be between about 5 and about 25 percent, alternatively between about 12 and about 18 percent and still further between about 14 and about 16 percent. The percent open area in an array ((30), (40), (50)) is the total surface area of the depressions as measured at the top surface (22) formed by the depressions divided by the total surface area of the top surface (22) of the portion of array being used with the quotient being multiplied by 100 to yield the percentage. Note that within an array if any portion of the area or an array chosen meets these parameters, the array as a whole is considered to meet these parameters.

When describing the dimensions of the apertures and depressions, in addition to open area of the individual apertures and depressions, the size of the apertures and depressions can be quantified in the context of the major axis of the particular aperture or depression. The “major axis” is the length of the longest interior line that can be drawn between two points on the edge of the aperture (or depression when measuring depressions as opposed to apertures) without intersecting or touching a third point on the edge of the aperture (or depression). The major axis of the aperture is measured across the aperture in the bottom of the depression. The major axis of the depression is measured at the top surface (22) of the fibrous nonwoven web (20) which in the product embodiments is the top surface of the topsheet (202). Thus, in the first array (30), the first aperture (32) will have a first major axis and the first depression (34) will have a first major axis. Likewise, the second aperture (42) and the second depression (44) in the second array (40) will each have a second major axis to distinguish them from the first major axes of the first array (30) and the third aperture (52) and the third depression (54) in the third array (50) will each have a third major axis.

Generally, the first major axis of the first apertures (32) in the first array (30) should be between about 0.3 and about 5.0 millimeters, alternatively between about 1.0 and about 2.0 millimeters. Generally and independent of the first array (30), the second major axis of the second apertures (42) in the second array (40) should be between about 0.3 and about 5.0 millimeters, alternatively between about 1.0 and about 3.0 millimeters and still further between about 1.2 and about 2.5 millimeters. Generally and independent of the first array (30) and the second array (40), the third major axis of the third apertures (52) in the third array (50) should be between about 0.3 and about 5.0 millimeters, alternatively between about 1.0 and about 3.0 millimeters and still further between about 1.0 and about 2.0 millimeters. Note that within an array if any portion of the area or an array chosen meets these parameters, the array as a whole is considered to meet these parameters.

As to all the apertures, note that the aperture sizes and shapes can be varied in many ways. In addition to different sized apertures from one array to another, the apertures in one array may have different sizes as well as different shapes. Further the shapes of the apertures can be of any shape or combination of organic and geometric shapes including, but not limited to, circles, ovals, rectangles, squares, diamonds, polygons and irregular-shaped apertures and this is true within an array or as between arrays. Organic shapes are shapes with a natural look and a flowing and curving appearance. For this reason, they are often also referred to as curvilinear shapes. Examples of organic shapes include the shapes of leaves, plants, and animals.

Generally, the first depressions (34) in the first array (30) formed by the embossing should have a first major axis in the top surface (22) of the fibrous nonwoven web (20) between about 0.3 and about 6 millimeters, alternatively between about 1.0 and about 3.5 millimeters and still further between about 1.0 and about 2.4 millimeters. Generally and independent of the first array (30), the second major axis of the second depressions (44) in the second array (40) should be between about 1.0 and about 8.0 millimeters, alternatively between about 2.0 and about 6.0 millimeters and still further between about 3.0 and about 5.0 millimeters. Generally and independent of the first array (30) and the second array (40), the third major axis of the third depressions (54) in the third array (50) should be between about 0.3 and about 6.0 millimeters, alternatively between about 1.0 and about 3.5 millimeters and still further between about 1.0 and about 2.4 millimeters. Note that within an array if any portion of the area or an array chosen meets these parameters, the array as a whole is considered to meet these parameters.

As to all the depressions formed by the embossing, note that the depression sizes and shapes can be varied in many ways. In addition to different sized depressions from one array to another, the depressions in one array may have different sizes as well as different shapes. Further the shapes of the depressions can be of any shape or combination of organic an geometric shapes including, but not limited to, circles, ovals, rectangles, squares, diamonds, polygons and irregular-shaped apertures and this is true within an array or as between arrays. Organic shapes are shapes with a natural look and a flowing and curving appearance. For this reason, they are often also referred to as curvilinear shapes. Examples of organic shapes include the shapes of leaves, plants, and animals.

Generally the basis weight of the fibrous nonwoven web (20) will be between about 20 and about 50 grams per square meter (gsm), alternatively between about 20 and about 35 gsm. This is especially true when the fibrous nonwoven web (20) is being employed as a topsheet or other layer for an absorbent article though basis weights outside these ranges are also possible.

In addition to the foregoing, the fibrous nonwoven webs (20) of the present invention were observed to have a number of desired functions including improved absorbency, improved breathability and a bulkier and thicker feel. The fibrous nonwoven webs (20) were also perceived to be more three-dimensional in design and aesthetically more feminine looking than conventional designs such as shown in FIG. 5 of the drawings. In processing and conversion into a finished product, the fibrous nonwoven webs of the present invention were found to convert better than the material shown in FIGS. 4A and 5. It was observed that the embossing pattern used in FIG. 6 had less deformation in both the machine direction (MD) and cross-machine (CD) direction than the conventional fabric and embossing pattern shown in FIG. 5 of the drawings during the forming and conversion processes and during use.

### Process and Apparatus

The equipment and process used to form the fibrous nonwoven web (20) resulted in a single step process wherein both the embossed depressions and the apertures were formed in each array in one step. This was accomplished by using a mating male and female roll with the male roll having the pattern shown, for example, in FIG. 6 of the drawings. While a two or multi-step process could be used, for indexing purposes the embossing step and pasturing step were done at the same time so that the apertures could be accurately placed within the bottoms of the depressions embossed into the fibrous nonwoven web (20).

The embossed depressions and apertures were formed by creating raised areas on the surface of the male role with aperturing pins located on the distal ends of the raised areas. In the case of the first depressions (34) and the third depressions (54) the raised areas were a generally circular design with a diameter, adjacent the base or proximal ends of the raised areas (adjacent the exterior surface of the male roll), of 1.6 millimeters. The raised areas tapered to a point and had a vertical height of 3.0 millimeters. An enlarged view of the resultant aperture and depression formed by these raised areas on the male roll is shown in FIG. 6B.

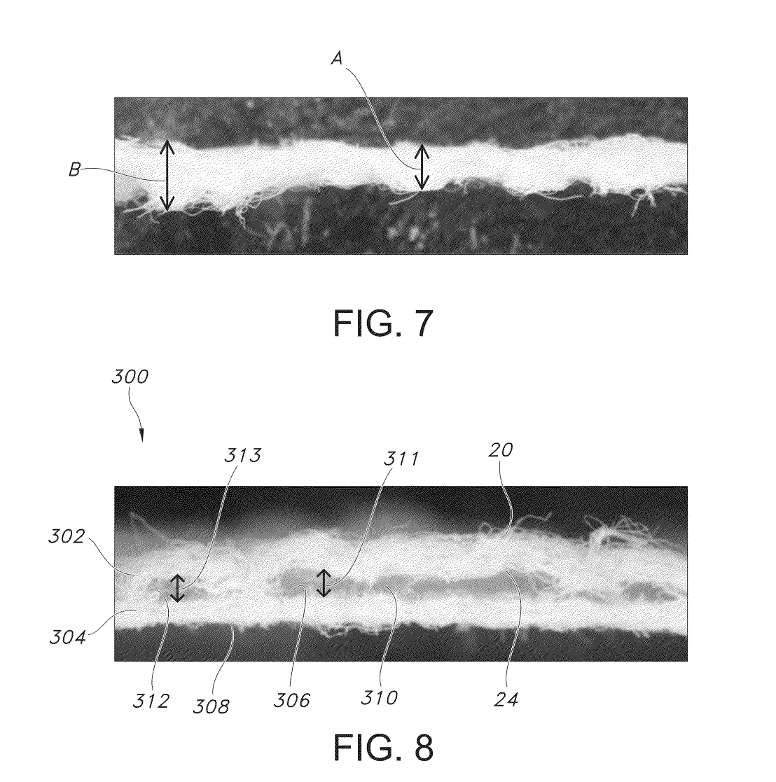

In the case of the second depressions (44) the raised areas were of a racetrack design with straight sides and curved ends with a length, adjacent the base or proximal ends of the raised areas (adjacent the exterior surface of the male roll), of 2.9 millimeters and a width of 1.7 millimeters. The raised areas tapered in size towards their distal ends and had a vertical height or 3.0 millimeters. The distal ends of the of the raised areas were capped with elongated knife-like edge with a width of approximately 1.2 millimeters. An enlarged view of the resultant aperture and depression formed by these raised areas on the male roll is shown in FIG. 6C. As can be seen from FIG. 7, the variance in the vertical height of the raised areas on the male roll in turn affects the depth of the depressions formed in the nonwoven web and the bulkiness of the resultant web. Thickness “A” in FIG. 7 is the result of the embossing and aperturing pins associated with the first array (30) of first apertures (32) and first depressions (34). Thickness “B” in FIG. 7 is the result of the embossing and aperturing pins associated with the second array (40) of second apertures (42) and second depressions (44) with the thickness “B” being thicker than the thickness “A”. Thickness “A” was 1.42 millimeters while thickness “B” was 0.95 millimeters—both measurements being taken under a no load condition. In certain embodiments, it is desirable that the thickness B be anywhere from about 30 percent to about 100 percent greater than the thickness of A. That is, it is desirable that the differential between the depths of the depressions in one array be anywhere from about 30 to about 100 percent greater or smaller than the depressions in another array.

Comparing the depressions and apertures of FIGS. 6B and 6C it can be seen that there was much less taper in the sidewalls of the first depressions (34) in FIG. 6B as compared to the side walls of the second depressions (44) in FIG. 6C. The second depressions (44) with their second apertures (42) in the second array (40) were deeper and provided more volume than the first depressions (34) and their first apertures (32) and the third depressions (54) and their third apertures (52) thus providing more support, bulk and integrity to the resultant fibrous nonwoven web (20). Conversely, the first apertures (32) and the third apertures (52) had more open area than the second apertures (42), thereby providing more fluid flow potential thereby making the overall fibrous nonwoven web (20) suitable in the context of accepting body exudates when the web (20) is being used in conjunction with absorbent articles (200). Not to be bound by a theory, the apertured and embossed fibrous nonwoven webs in the present application are believed to work to control the deformation of the topsheet to drive controlled movement of both air and fluid through the material and down into the product as it is worn. In this regard, see U.S. Pat. No. 7,145,054 to Zander et al. which is incorporated herein by reference in its entirety.

Due to the difference in vertical height of the raised areas on the male roll, at least a portion of some of the apertures from the first (30), second (40) and third (50) array will terminate in or adjacent and help define the bottom surface (24) of the fibrous nonwoven web (20). Further, due to the fact that other raised areas on the male roll will have a shorter vertical height, at least a portion of one of the other of the apertures in the first (30), second (40) and third (50) arrays will terminate and therefore be located intermediate the top surface (22) and the bottom surface (24) of the fibrous nonwoven web (20). This in turn helps with air circulation below the bottom surface (24) of the fibrous nonwoven web (20). In addition, that portion of the apertures that are intermediate the top surface (22) and bottom surface (24) will be located above that portion of the apertures that reside in or adjacent the bottom surfaced (24).

The male and female rolls were both driven at the same speed by electric motors. If desired, either or both of the male and female rolls can be heated or cooled to improve the aperturing and embossing process.

While performing the embossing and aperturing in a single step was a preferred process, it is also possible to perform the various steps separately. For example, the embossing could be done first and the aperturing second or vice versa. Additionally, the embossing and aperturing steps for each of the arrays could be done separately though this is considered to be a more complicated process due to indexing and other issues.

Once the fibrous nonwoven web (20) has been formed, it can be directed immediately into a production process such as the formation of a personal care absorbent article 200 such as is shown in FIG. 1. Alternatively, it may be wound up in roll form for subsequent processing.

In addition to being formed either in-line or off-line, the fibrous nonwoven web (20) may be subjected to further processing such as, for example, formation into a fibrous nonwoven laminate (300) as described herein and shown in the photo of FIG. 8 having a first nonwoven web (302) and a second nonwoven web (304), either or both of which may be formed from the fibrous nonwoven web (20) as described herein. For example, the first fibrous nonwoven web (302) may be formed from the fibrous nonwoven web (20) and it may be unwound and laid upon the upper surface (306) of the second nonwoven web (304) so as to form the fibrous nonwoven web (300). If desired, the two webs (302 and 304) may be joined to one another such as, for example, by the use of adhesive, thermal bonding, ultrasonic bond or hydroentangling. The fibrous nonwoven laminate (300) so formed may have one or more first air gaps (310) and one or more second air gaps (312) formed between the bottom surface (24) of the first fibrous nonwoven web (302) formed from the fibrous nonwoven web (20) and the upper surface (306) of the second fibrous nonwoven web (304). This is because the second depressions (44) in the second array (40) are deeper than, for example, the first depressions (34) in the first array (30) and the third depressions (54) in the third array (50). The wider air gap (310) corresponds to the gap underneath the leaf-like design of the first array (30) and the narrower air gap (312) corresponds to the gap underneath the sinusoidal pattern of the third array (50).

The first air gaps (310) will define a first vertical height (311) and the second air gaps (312) will define a second vertical height (313). Depending on the vertical heights of the first (34), second (44) and third (54) depressions, these vertical heights (311) and (313) may be equal to, greater than or less than one another. In addition, when one or more of the arrays ((30), (40), (50)) are formed in the sinusoidal or other continuous form such as is shown in the drawings, one or more of the air gaps ((310), (312)) may also form air channels that extend along all or a portion of the laminate (300). A laminate (300) as described above can also be used as a topsheet (202) or as one of the other layers for an absorbent article (200). The advantage of this material is that it is able to provide increased thickness or bulk as the same basis weight as with other more two-dimensional materials while also improving breathability and decreasing skin contact on the top surface (22) of the web (20) due to the open area provided by the arrays of apertures and depressions.

While the fibrous nonwoven web (20) has been described herein primarily in the context of its use with absorbent articles, wipes and cleaning products, it should be appreciated that it can be used in any applications where a material with such arrays of apertures and depressions can provide beneficial results either alone or as a part or component of an overall product.

It will be recognized that the present invention is capable of many modifications and variations without departing from the spirit and scope thereof. Accordingly, the detailed description and examples set forth herein are meant to be illustrative only and are not intended to limit, in any manner, the scope of the invention as set forth in the appended claims.

What is claimed is:

1. A fibrous nonwoven web defining a top surface and a bottom surface and comprising a first array of first apertures, each of said first apertures residing in a respective first depression in said web, said first depression having a first depression depth, a first depression density, a first depression open area and a first depression shape, said first array of first apertures defining a first aperture density, a first aperture open area and a first aperture shape;
a second array of second apertures, each of said second apertures residing in a respective second depression in said web, said second depression having a second depression depth, a second depression density, a second depression open area and a second depression shape, said second array of second apertures defining a second aperture density, a second aperture open area and a second aperture shape, said second array of second apertures at least partially surrounding said first array, said second aperture depth being different from said first aperture depth, and at least one of said second aperture density and said second aperture open area respectively being different from said first aperture density and said first aperture open area; and a third array of third apertures, each of said third apertures residing in a respective third depression in said web, said third depression having a third depression depth, a third depression density, a third depression open area and a third depression shape, said third array of third apertures defining a third aperture density, a third aperture open area and a third aperture shape, said second array at least partially separating said first array from said third array.

2. The fibrous nonwoven web of claim 1 wherein at least a portion of one of said first, second and third apertures is located in or adjacent said bottom surface and at least another portion of one of the other of said first, second and third apertures is located intermediate said top surface and said bottom surface.

3. The fibrous nonwoven web of claim 2 wherein at least one of said first aperture density and said first aperture open area is respectively greater than said second aperture density and said second aperture open area.

4. The fibrous nonwoven web of claim 3 wherein said first apertures each define a first major axis and said second apertures each define a second major axis, said second major axis being greater than said first major axis.

5. An absorbent article comprising a topsheet, a backsheet and an absorbent core disposed between said topsheet and said backsheet and optionally one or more additional layers disposed between said topsheet and said absorbent core, wherein at least a portion of said absorbent article comprises the fibrous nonwoven web of claim 1.

6. An absorbent article comprising a topsheet, a backsheet and an absorbent core disposed between said topsheet and said backsheet and optionally one or more additional layers disposed between said topsheet and said absorbent core, wherein at least a portion of said absorbent article comprises said fibrous nonwoven web of claim 4.

7. The absorbent article of claim 5 wherein at least a portion of said topsheet comprises said fibrous nonwoven web.

8. The absorbent article of claim 6 wherein at least a portion of said topsheet comprises said fibrous nonwoven web.

9. The absorbent article of claim 8 wherein said article has a layer subjacent said topsheet which is a darker color than said topsheet.

10. A topsheet for an absorbent article comprising the fibrous nonwoven web of claim 1.

11. A topsheet for an absorbent article comprising the fibrous nonwoven web of claim 4.

12. The fibrous nonwoven web of claim 1 wherein said second array has a sinusoidal shape.

13. The fibrous nonwoven web of claim 12 wherein said first array has a noncircular shape.

14. The fibrous nonwoven web of claim 13 wherein said third array has a sinusoidal shape.

15. The fibrous nonwoven web of claim 14 wherein said first, second and third arrays form a repeating pattern and said second and third arrays are in phase with one another.

16. The fibrous nonwoven web of claim 14 wherein said first, second and third arrays form a repeating pattern and said second and third arrays are out-of-phase with one another.

17. The fibrous nonwoven web of claim 1 wherein at least one of said third aperture density and said third aperture open area is respectively greater than said second aperture density and said second aperture open area.

18. A fibrous nonwoven laminate comprising a first fibrous nonwoven web and a second fibrous nonwoven web, said second fibrous nonwoven web defining an upper surface and a lower surface, said first fibrous nonwoven web comprising said fibrous nonwoven web of claim 2 with said bottom surface of said fibrous nonwoven web positioned adjacent said upper surface of said second fibrous nonwoven web whereby a first air gap defining a first vertical height and a second air gap defining a second vertical height are formed between said first and second fibrous nonwoven webs,

19. The fibrous nonwoven web of claim 1 wherein said second array completely surrounds said first array and wherein at least one of said second aperture open area and said second depression open area is respectively less than said first aperture open area and said first depression open area.

20. The fibrous nonwoven web of claim 1 wherein said second array completely surrounds said first array and wherein at least one of said second aperture shape and said second depression shape is respectively different from said first aperture shape and said first depression shape.
